(19) United States
(12) Patent Application Publication
(10) Pub. No.: US 2003/0157533 A1
(43) Pub. Date: Aug. 21, 2003

(54) Nucleic acid labeling by Thermoanaerobacter thermohydrosulfuricus DNA polymerase I variants

(76) Inventors: Maria Davis, Princeton, NJ (US); Chockalingam Palaniappan, Belle Mead, NJ (US)

Correspondence Address: Amersham Biosciences Corp, 800 Centennial Avenue, Piscataway, NJ, 08855, US

(21) Appl. No.: 10/326,040

(22) Filed: Dec. 20, 2002

Related U.S. Application Data
(60) Provisional application No. 60/343,023, filed on Dec. 20, 2001.

(51) Int. Cl. C12Q 1/68; C07H 21/04; C12P 19/34; C12N 9/22; C12N 1/21; C12P 21/02; C12N 15/74

(52) U.S. Cl. 435/6; 435/69.1; 435/199; 435/252.3; 435/91.2; 435/320.1; 536/23.2

(57) ABSTRACT

An enzymatically active DNA polymerase or active fragment thereof, having at least 80% identity in its amino acid sequence to the DNA polymerase of Thermoanaerobacter thermohydrosulfuricus or fragment thereof, and having an amino acid alteration at position 720 in Tts Pol I or at position 426 in ΔTts Pol I or at a homologous position defined with respect to Tts DNA ploymerase I, having improved incorporation of nucleotide analogs and natural bases during DNA synthesis compared to unaltered enzyme.

### BACKGROUND OF THE INVENTION

1. Field of the Invention

Thermoanaerobacter thermohydrosulfuricus (Tts) DNA polymerase has been cloned and expressed in E. coli and purified. A U.S patent (U.S. Pat. No. 5,744,312) has been recently issued to Amersham Life Science, Inc. A significant property of this polymerase is its ability to catalyze RNA-dependent DNA polymerase activity, reverse transcriptase activity (U.S. Pat. No. 5,744,312), in addition to its DNA dependent DNA polymerase. This polymerase performs optimally at a broad temperature range from 37-65 C. with maximal activity at 60 C. These activities combined with thermostability of the enzyme offer several benefits as discussed below. Several different variants of the enzyme have been generated for utility in DNA sequencing, for use in first-strand cDNA synthesis, RT-PCR and for strand displacement amplification.

2. Description of Related Art

DNA polymerases and Reverse Transcriptases (RTs) isolated from various organisms ranging from bacteria, viruses, archaebacteria are being successfully used in the field of molecular biology for various applications. The growth temperatures for these organisms could range from extremely low to high. Applications of enzymes derived from the organisms range from cloning, polymerase chain reaction (PCR) (U.S. Pat. No. 4,683,195, Mullis et. al.), DNA sequencing, mutagenesis, genomic library construction, and nucleic acid labeling such as cDNA labeling for micro and macro arrays.

DNA Polymerases discriminate against the incorporation of unnatural bases during DNA synthesis. Most naturally occurring DNA polymerases also do not employ RNA as a template molecule. However, the natural template for a reverse transcriptase is both RNA and DNA. The natural building blocks for DNA polymerases and RTs are the four deoxy ribonucleotides (dATP, dGTP, dCTP and dTTP). Most naturally occurring polymerases and reverse transcriptases exhibit poor incorporation efficiencies towards most nucleotide analogs. The analogs could be any variants of naturally occurring dNTPs, such as ddNTPs, rNTPs, conjugates (dye or otherwise) of dNTPs and ddNTPs. This selection is important in the survival of the host. Frequent incorporation of non-natural bases would hamper subsequent rounds of replication resulting in the ultimate death of the organism. If and when polymerases do incorporate non-natural bases in their host, it is under extreme conditions that would lead to the ultimate survival of the organism. Such events however lead to mutations in the organism that may be needed for survival under extreme conditions. Therefore it is not unusual that native polymerases, having wild type amino acid sequence, either isolated directly from the host or by recombinant means exhibit a discriminatory effect towards non-natural nucleotides. Nevertheless, under very high concentrations of the analogs, native polymerases do incorporate these analogs during DNA synthesis albeit poorly. This feature is currently being exploited in all applications that use DNA polymerases or RTs for nucleic acid labeling. Consequently, the specific activity of the probe made using the naturally occurring polymerases or RTs is generally low. The current approaches to using natural enzymes for labeling encounter numerous technical difficulties. For example, incorporation of fluorescently labeled nucleotides by these naturally occurring enzymes can only be marginally improved by using excessive amounts of these labeled nucleotides in the reaction. But this imposes a different set of problems. It is generally difficult to remove the unused excess labeled nucleotides after the reaction, imposing serious problems with respect to poor signal to noise ratios. Additionally, a large amount of usually rare raw material is used to achieve marginal labeling. Apart from these problems, there is also sacrifice in the yield of the total probe generated. This is attributed to the discrimination by wild type polymerases and RTs to extend from an incorporated dNTP analog, such as a dye-dNTP. This again is a built-in feature of wild type polymerases and reverse transcriptases.

Higher specific activity probes are useful in multiple applications. This requires a facile addition of dye-dNMP followed by subsequent extension. Repeated rounds of addition of dye-dNMP and extension results in the formation of probes with higher specific activity. Since, naturally occurring polymerases and RTs are discriminatory to both addition and extension of a dNTP analog or dye-dNTP, the probes generated are of low specific activity.

As the above discussion suggests, a way of altering the natural properties of polymerases for better incorporation of nucleotide analogs during DNA synthesis, is desirable. For example, an improved ability to incorporate labeled nucleotides in various nucleic acid applications such as rolling circle amplification and single nucleotide polymorphism detection would be useful. This concern is addressed in greater detail below.

### SUMMARY OF THE INVENTION

Accordingly, it is the object of the invention to provide an enzymatically active DNA polymerase having improved incorporation of nucleotide analogs and natural bases during DNA synthesis and a method of incorporating dye labeled dNTP's using the DNA polymerase or an active fragment thereof. It is a further object of the invention to provide a method of utilizing the DNA polymerase for performing direct RNA sequencing and to provide kits for labeling a polynucleotide from a DNA or RNA template with a DNA or RNA primer comprising the DNA polymerase.

The objectives are met by the present invention, which relates in one aspect to a DNA polymerase or active fragment thereof. The DNA polymerse or active fragment thereof, has at least 80% identity in its amino acid sequence to the DNA polymerase of Thermoanaerobacter thermohydrosulfuricus or a fragment thereof, and has an amino acid alteration at position 720 in Tts Pol I or at position 426 in ΔTts or at a homologous position defined with respect to Tts DNA polymerase, and has improved incorporation of nucleotide anaologs and natural bases during DNA synthesis, as compared to unaltered enzyme. In one embodiment the nucleotide analogs are dNTP, ddNTP and rNTP analogs. In a second embodiment the dNTP, ddNTP and rNTP analogs are dye-conjugated or biotin-conjugated. In a third embodiment the dye in the dye-conjugated nucleotide analogs is a rhodamine or Cyanine derivative dye. In a fourth embodiment the rhodamine dye is R110, R6G, TMR or Rox. In a fifth embodiment the Cyanine derivative dye is Cy3, Cy3.5, Cy5.0 or Cy5.5. In a sixth embodiment the DNA polymerase has the asparatate at position 720 in Tts Pol I or at position 426 in ΔTts Pol I, replaced with agrinine.

A related aspect of the invention relates to a method of utilizing the DNA polymerase of Thermoanaerobacter thermohydrosulfuricus or a fragment thereof, having an amino acid alteration at position 720 in Tts Pol I or at position 426 in ΔTts or at a homologous position defined with respect to Tts DNA polymerase, having improved incorporation of nucleotide analogs and natural bases during DNA synthesis, as compared to unaltered enzyme, for incorporating Cy3 and Cy5 dye conjugated dNTP's across a range of reaction temperatures form 37-65° C.

In a further aspect, the invention relates to a method of utilizing the DNA polymerase for performing direct RNA sequencing, while a further aspect relates to providing kits for labeling a polynucleotide from a DNA or RNA template with a DNA or RNA primer comprising the DNA polymerase.

The above objects and features of the invention will become more fully apparent when the following detailed description of the invention is read in conjunction with the accompanying figures.

### BRIEF DESCRIPTION OF DRAWINGS

FIG. 1. (SEQ ID No. 1) is the Amino acid sequence of the full-length of Tts DNA polymerase I. A full-length recombinant form of the enzyme, harboring both the native 5′-3′ DNA template mediated DNA polymerase function and 5′-3′ exonuclease. (Covered under U.S. Pat. No. 5,744,312) serves as a reference amino acid sequence. In addition, the enzyme harbors reverse transcriptase activity.

FIG. 1A. (SEQ ID No. 2) is the DNA sequence of the full-length of Tts DNA polymerase I (Covered under U.S. Pat. No. 5,744,312)

FIG. 2. (SEQ ID No. 3) is the amino acid sequence of the ΔTts DNA polymerase I. A 5′-3′ exonuclease deficient (exo−) form of the enzyme, with a truncation at the amino-terminus. (Covered under U.S. Pat. No. 5,744,312). Blocked portion represents the region of the deleted amino acids from the full-length version of the enzyme.

FIG. 2A. DNA sequence of the ΔTts DNA polymerase I. (SEQ ID No. 4). (Covered under U.S. Pat. No. 5,744,312)

FIG. 3. Amino acid sequence of the F412Y variant of the ΔTts DNA polymerase I. (Covered under U.S. Pat. No. 5,744,312). Blocked portion represents the region of the deleted amino acids from the full-length version of the enzyme (Position 412 in ΔTts corresponds to 706 in full-length enzyme, phenylalanine in this position is implicated in discrimination towards ddNTP. The F412Y change facilitates easy incorporation of ddNTP. (SEQ ID No. 5)

FIG. 3A. DNA sequence of the F412Y variant of the ΔTts DNA polymerase I. (SEQ ID No. 6). (Covered under U.S. Pat. No. 5,744,312)

FIG. 4. Amino acid sequence of the ΔTtsF412YD426R variant polymerase. Blocked portion is the deleted amino acids from the full-length version of the enzyme. Position 412 in ΔTts corresponds to 706 in full-length enzyme, phenylalanine in this position is implicated in discrimination towards ddNTP. Position 426 in ΔTts corresponds to 720 in full-length enzyme. Discrimination towards both incorporation and extension of a dye conjugates of dNTP, rNTP or ddNTP is governed by aspartate residue at this position. Mutation was generated by oligonucleotide based site-directed mutagenesis technique to introduce a Δ Tts D426R change in the Tts F412Y background. (SEQ ID No. 7)

FIG. 5. Amino acid sequence of the ΔTts D426R polymerase. Blocked portion is the deleted amino acids from the full-length version of the enzyme. Position 426 in ΔTts corresponds to 720 in full-length enzyme. Discrimination towards both incorporation and extension of a dye conjugates of dNTP, rNTP or ddNTP is governed by aspartate residue at this position. (SEQ ID No. 8)

FIG. 6. Alignment of wild type Pol I sequences from different microorganisms. Homologous positions around the “finger region” of Polymerases of Pol I family are shown here. Richardson, “DNA polymerase from Escherichia coli,” Procedures in Nucleic Acid Research, Cantoni and Davies editors, Harper and Row, New York, pp. 263-276 (1966). Scopes, Protein Purification, Springer-Verlag, New York, N.Y., pp. 46-48 (1994).

FIG. 7. Improved incorporation of Dye (Cy 3.5)-dCTP and dCTP by ΔTts D426R form of ΔTts Pol I.

FIG. 8. Direct RNA sequencing by AMV RT, ΔTts and ΔTts F412Y Pol I.

FIG. 9. ΔTts F412YD426R performance in cDNA labeling using Cy3 and Cy5-dCTP and utility in microarray applications.

FIG. 10. ΔTts F412YD426R performance in cDNA labeling using Cy3 and Cy5-dUTP and utility in microarray applications.

FIG. 11. Usefulness of ΔTts F412Y or ΔTtsF412YD426R Pol I in single nucleotide primer extension (SnuPE), using RNA templates. Incorporation of dye labeled or unlabeled ddA, ddT, ddG and ddC is demonstrated here.

FIG. 12. Utility of ΔTts DNA polymerase in Rolling Circle Amplification reaction

FIG. 13. Incorporation of dye labeled nucleotide during DNA dependent DNA synthesis ΔTts, ΔTts F412Y, ΔTts F412YD426R.

FIGS. 14a & b. ΔTtsF412YD426R Performance in cDNA labeling using Cy3/Cy5-dCTP; demonstration of accurate determination of gene expression over a wide reaction temperature range (FIGS. 14a and b).

### DETAILED DESCRIPTION OF THE INVENTION

The present invention discloses the utility of native DNA pol I and variant forms of DNA Pol I of Thermoanaerobacter thermohydrosulfuricus for nucleic acid labeling by fluorescent nucleotide analogs. Utility in applications such as cDNA labeling, rolling circle amplification, RNA sequencing and single nucleotide primer extension on RNA is also covered.

In this present invention we have found ways of altering the natural properties of polymerases for better incorporation of nucleotide analogs during DNA synthesis. Described here are modifications that can be introduced to the naturally occurring polymerases/reverse transcriptases to facilitate incorporation of fluorescent labeled nucleotides. The present invention identifies a single amino acid residue in polymerases that is responsible for improved incorporation of certain nucleotide analogs. A change in amino acid residue results in a profound increase in the ability of the enzyme for incorporation and extension from dye labeled nucleotides. This feature is useful in any nucleic acid application that employs fluorescent labeling by incorporation of nucleotide analog by a DNA polymerase. Such applications include labeling during DNA synthesis in various applications such as microarray analysis of gene expression. We also show the utility of some variants of the enzyme in direct RNA sequencing, rolling circle amplification, single nucleotide polymorphism detection (SNP) by single nucleotide primer extension utilizing either DNA or RNA templates. This invention relates to the wild type and mutant forms of the enzymes and their DNA sequence and amino acid sequence and the vectors that are used to generate them.

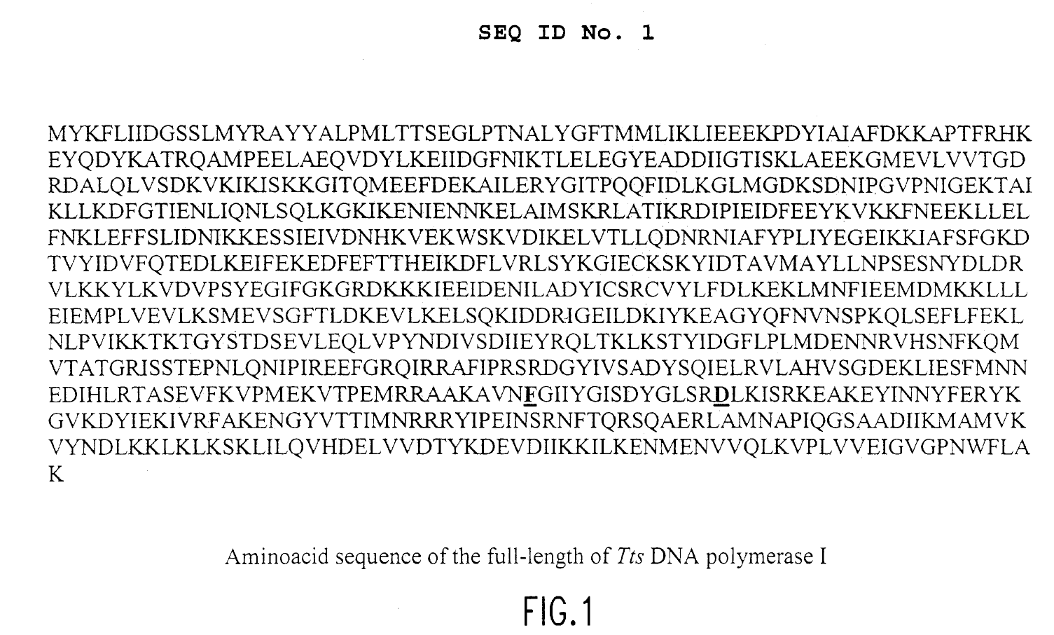

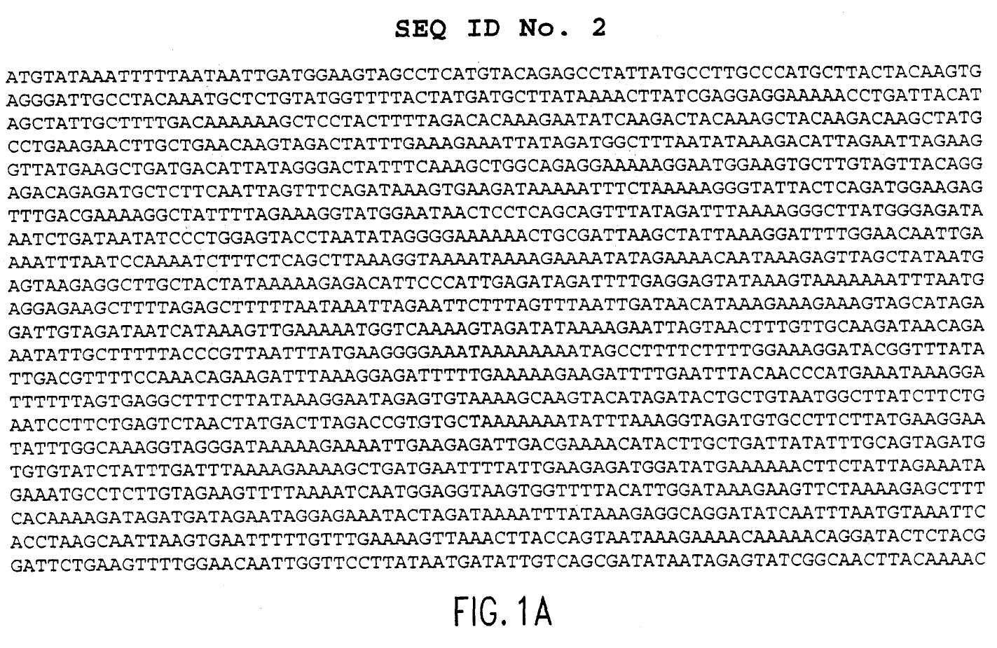

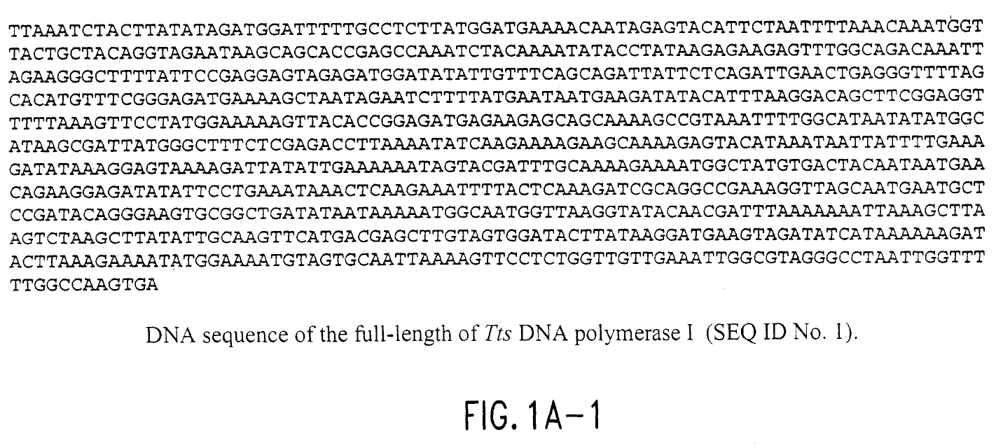

The first aspect of the invention relates to the generation and purification of a variant form of the native DNA Pol I of Thermoanaerobacter thermohydrosulfuricus and of sequences of polymerases that are at least 80% amino acid sequence identity as shown in FIG. 1 (U.S. Pat. No. 5,744,312).

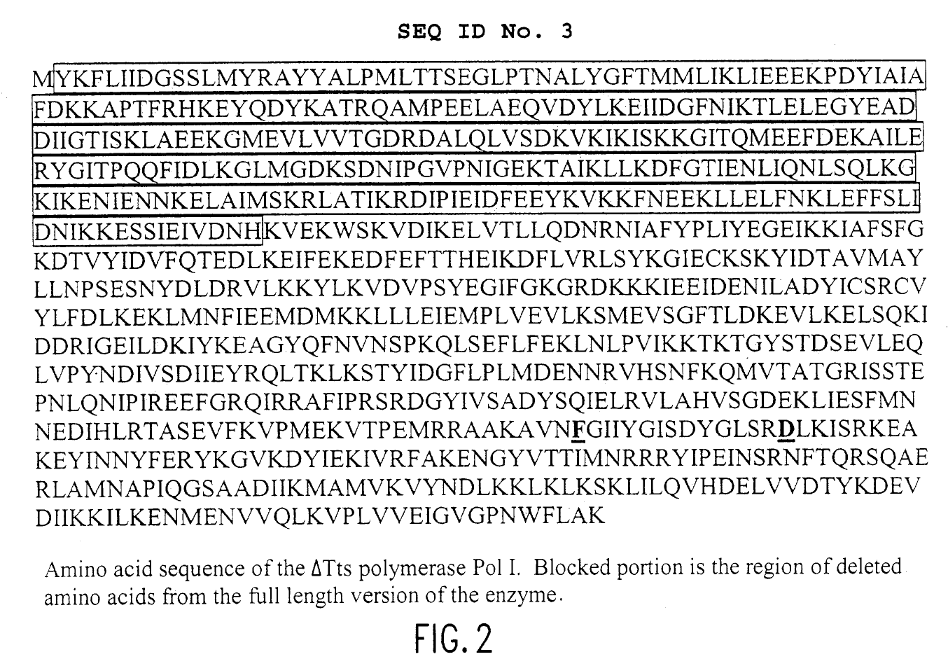

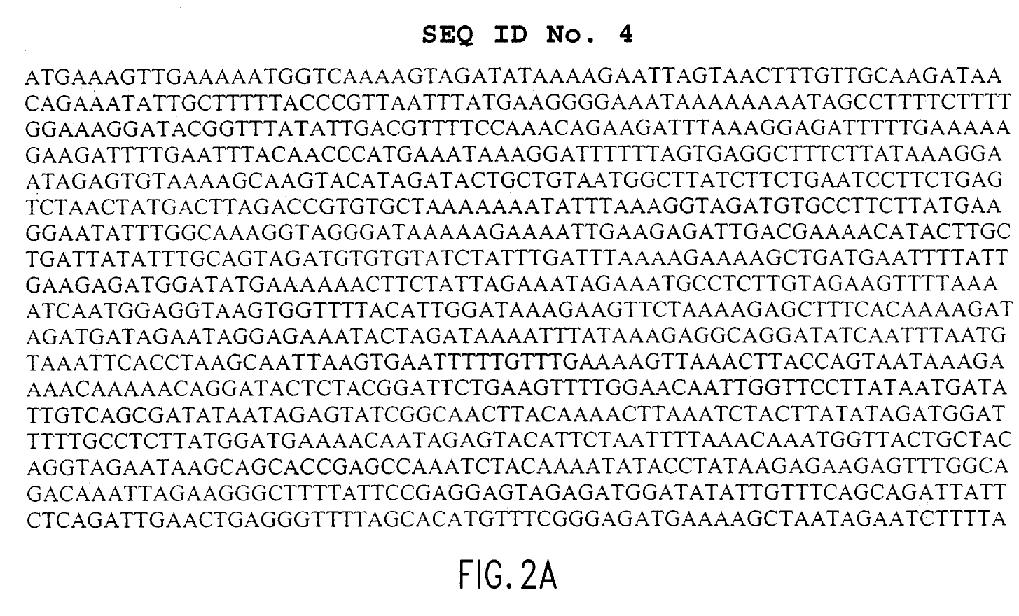

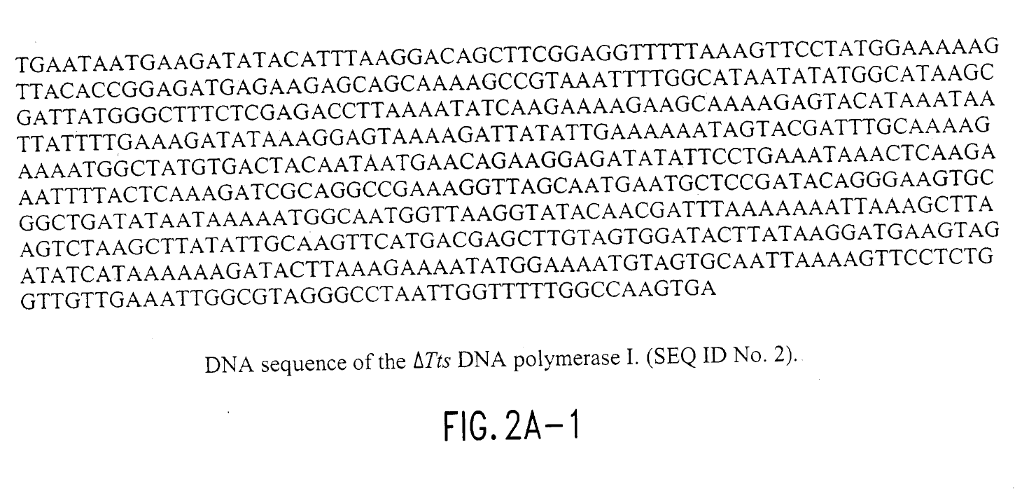

FIG. 1 shows the reference sequence of the amino acid encoded by the genomic DNA between positions 1056-3674 of the Tts revealed in patent (U.S. Pat. No. 5,744,312) (SEQ ID No. 1). Enzymes have been engineered in the previous disclosure to abolish an associated 5′-3′ exonuclease function in the native enzyme and is shown here as reference sequence in FIG. 2.

For ease of reference FIGS. 1, 2 and 3 are illustrated here (covered under U.S. Pat. No. 5,744,312). FIG. 1 is a full-length recombinant form of the enzyme, harboring both the native 5′-3′ DNA template mediated DNA polymerase function and 5′-3′ exonuclease. (covered under U.S. Pat. No. 5,744,312) serves as a reference amino acid sequence as shown in FIG. 1. The full-length version of the enzyme henceforth in this document will be referred to as Tts DNA Pol I.

FIG. 2 is a 5′-3′ exonuclease deficient (exo−) form of the enzyme, with a truncation at the amino-terminus. (covered under U.S. Pat. No. 5,744,312). Henceforth this form of the enzyme will be referred to as ΔTts enzyme. The numbering of amino acids for truncated form of the enzyme begins with the first amino acid of the truncated form. Additionally in some instances numbering of amino acids in this document is also indicated on non-truncated full-length version of the enzyme for easy comparison.

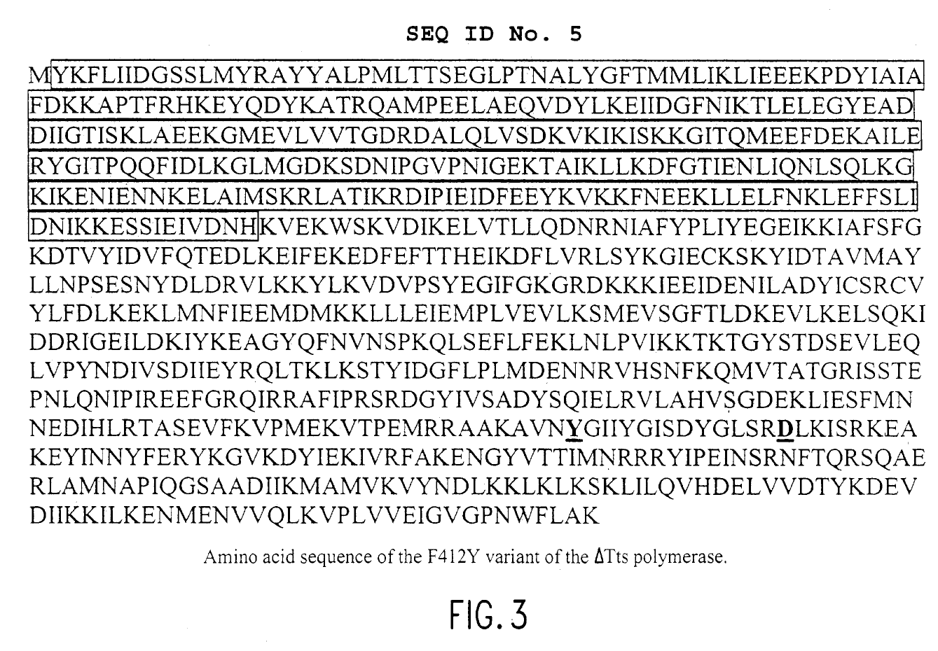

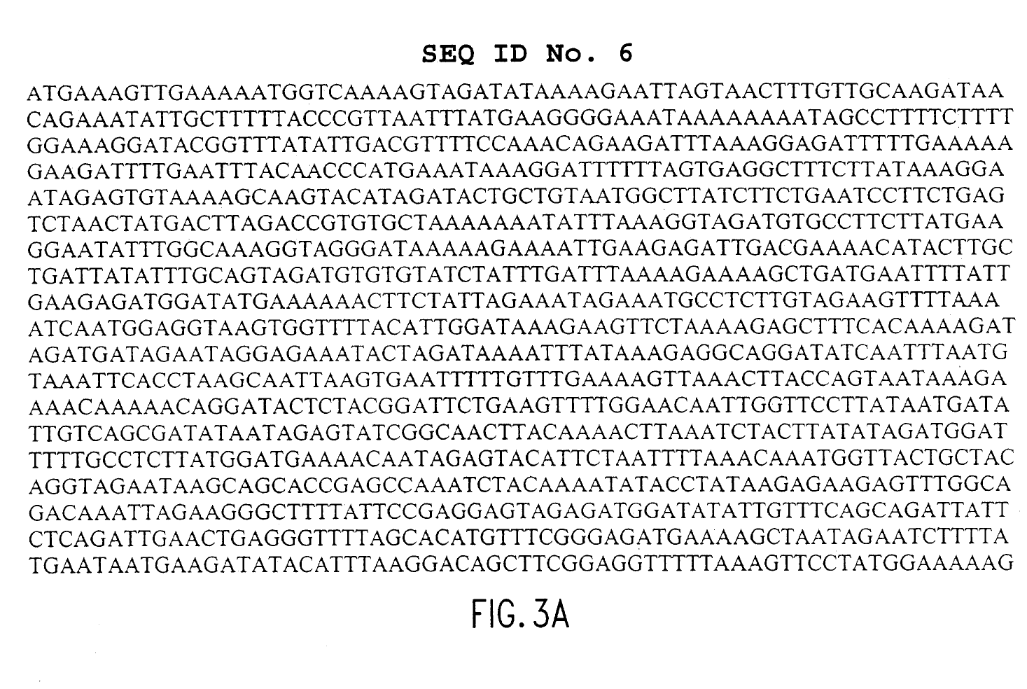

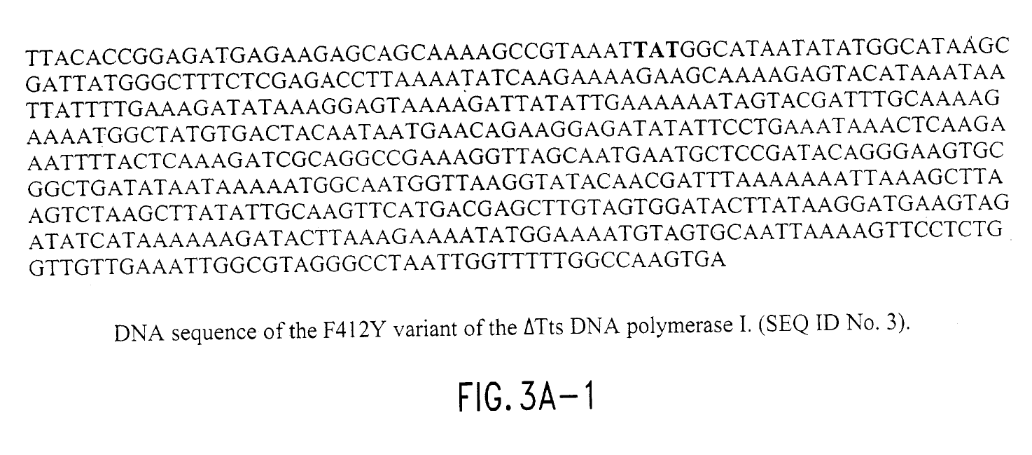

FIG. 3 is an exonulease deficient truncated version form of the enzyme with an F (phenlyalanine) to Y (Tyrosine) change in the O-helix region at position 412, and is shown for reference (covered under U.S. Pat. No. 5,744,312)

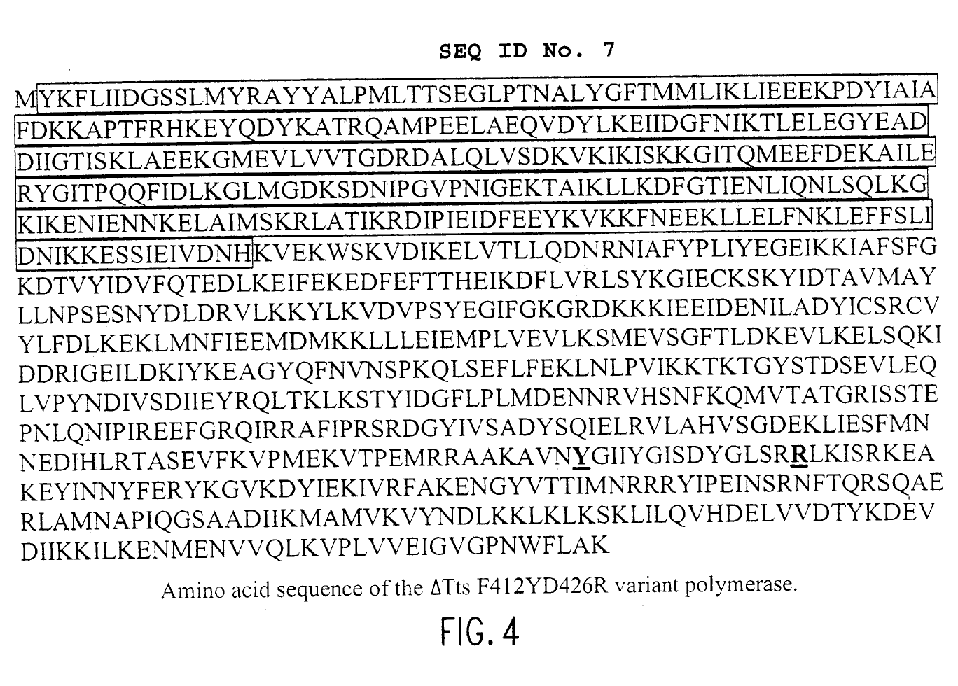

FIG. 4 is the enzyme showing the introduction of a point mutation altering the Aspartate (D) residue at 426 to Arginine (R) in ΔTtsF412Y form of the enzyme. This form of the enzyme henceforth will be referred to as ΔTtsF412YD420R.

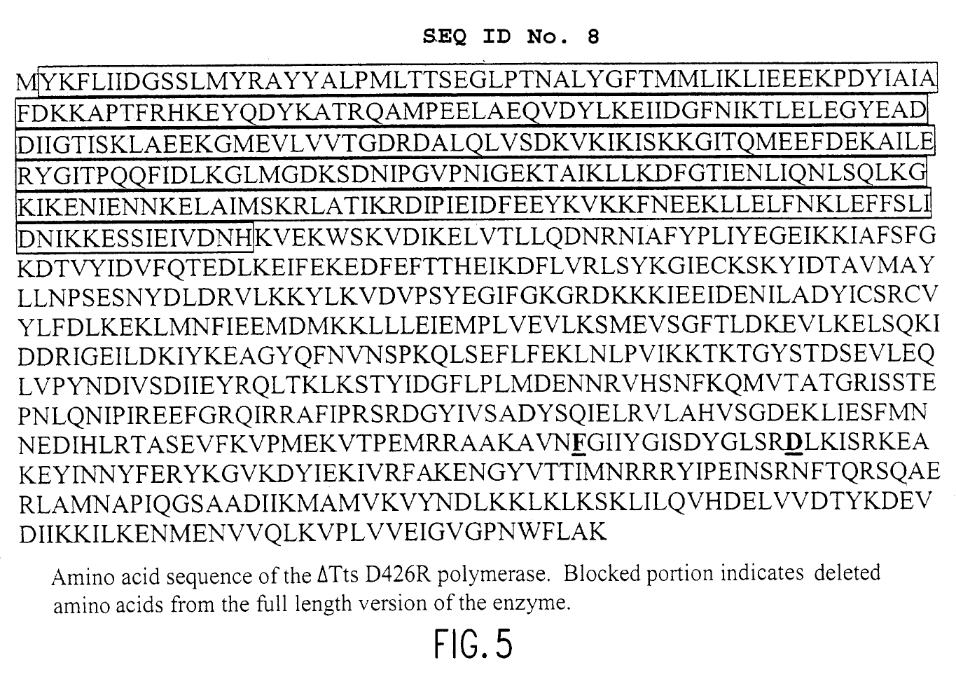

FIG. 5 shows another form of enzyme referred to as ΔTtsD426R by reversing the tyrosine (Y) residue at 412 back to phenylalanine (F) of ΔTtsF412Y form of the enzyme. Single letter amino acids are according to conventional codes used in the literature.

U.S. Pat. No. 5,744,312 shows utility for the native Tts, ΔTts, ΔTtsF412Y in applications ranging from cDNA preparation, strand displacement amplification (Walker et al., “Isothermal in vitro amplification of DNA by a restriction enzyme/DNA polymerase system,” Proc. Natl. Acad. Sci. USA 89:392-396 (1992)) and DNA sequencing.

The present application shows the utility of various forms of Tts enzyme in the incorporation of non-natural base analogs during DNA synthesis. Some examples are the incorporation of either unlabeled or dye-labeled versions of dNTPs, ddNTPs and rNTPs. DNA synthesis can be either DNA template mediated (DNA polymerase activity) or RNA template mediated (reverse transcriptase activity). DNA or RNA template-mediated cDNA probes are increasingly in demand for microarray applications. This invention demonstrates the utility of the enzyme variants in nucleic acid labeling during DNA synthesis with particular emphasis on microarray applications for gene expression studies.

Incorporation of ddNTP or ddNTP analogs is extremely useful in applications such as DNA or RNA sequencing. Herein it is demonstrated that ΔTtsF412Y and ΔTtsF412YD426R forms of the enzyme holds great promise for applications such as direct RNA sequencing and situations where single nucleotide primer extension is monitored. For example it can be seen that the F412Y variant is capable of generating excellent sequence information from short stretches of RNA compared to retroviral reverse transcriptases. Experimental results are also presented to document the utility of some enzyme variants in single nucleotide primer extension (SnuPE) applications for interrogation of target sequence of DNA or RNA backbone.

This finding is useful in applications that involve single polymorphism detection, mutation detection in DNA or RNA. Direct mutation detection at RNA level is useful in many respects. An example of such an application would be to determine drug resistance mutations in Human Immunodeficiency viral (HIV) RNA from patients undergoing drug treatments. Resistance mutations to HIV reverse transcriptase and protease inhibitors are attributed directly to mutations in the genes encoding these proteins in the RNA genome. Additionally, in humans and higher organisms improper splicing of RNA leading to defective mRNA is implicated in major disorders. Direct RNA sequencing of limited stretch such RNA or direct detection of improperly spliced RNA by mutation detection using SnuPE is feasible with the ΔTtsF412Y or ΔTtsF412RD426R variants. These would be different from current approaches that are being followed. Since retroviral RTs are not good sequencing enzymes, in current approaches a RT-PCR step is required before sequencing is undertaken.

In addition to mutation detection, Tts Pol I variants can be used for estimating RNA copy number. This has value in HIV research or gene expression studies. The enzyme's ability to incorporate dye-terminators and its potential for incorporating dye-labeled dNTP and ddNTP during cDNA synthesis can be capitalized on for estimating copy number of HIV-RNA, hence for estimating virus titer. This property of dye-labeled nucleotide incorporation by ΔTtsD426R would also be useful in mRNA quantification and gene expression studies on micro or macroarrays. The alternative strategies that are currently being employed: 1) quantitative RT-PCR used for estimating viral RNA and mRNA. 2) Branched DNA/nuclease excision for viral and mRNA quantitation.

The utility of Tts enzyme variants in strand displacement amplifications such as Rolling Circle Amplification (RCA) is also demonstrated in this invention.

### EXAMPLES

The following examples serve to illustrate the utility of the subject DNA polymerases and are for illustration purposes only and should not be used in any way to limit the appended claims.

### Example 1

Generation of ΔTts F412YD426R Variant (FIG. 4)

The expression vector PLS-3 harboring ΔTts F412Y variant disclosed in U.S. Pat. No. 5,744,312 served as a starting plasmid for this invention. Primers were designed to alter the codon encoding the residue 426 of ΔTts pol I from asparate to arginine. A forward primer of sequence “gggctttctcgacgccttaaaatatca” (SEQ ID No. 9)(encoding positions 422 to 430) and a complementary sequence was employed to introduce the intended point mutation. The new codon used for amino acid R was “cgc”. The primers were annealed and a cycling reaction with Pfu DNA polymerase was carried out in the presence of all four dNTPs to generate new strands. The final product was used in transformation of E. coli strain and colonies were screened individually by DNA sequencing to select for clones with desired mutation.

### Example 2

Generation of ΔTts D426R Variant (FIG. 5)

A clone containing the plasmid with ΔTts F412YD426R variant shown in example 1 above served as a starting material for the generation of ΔTts D426R variant. A strategy similar to above was employed. Two primers complementary to each other were designed to introduce the intended original phenylalanine “F” residue at position 412 of ΔTts F412YD426R. A forward primer GCCGTAAATTTTGGCATAATATATGGC (SEQ ID No. 10)(to span positions 409 to 417 of the ΔTts F412YD426R polymerase) and a complementary sequence was employed to change “Y” residue were designed. A codon “TTT” for phenylalanine was employed to engineer the change.

### Example 3

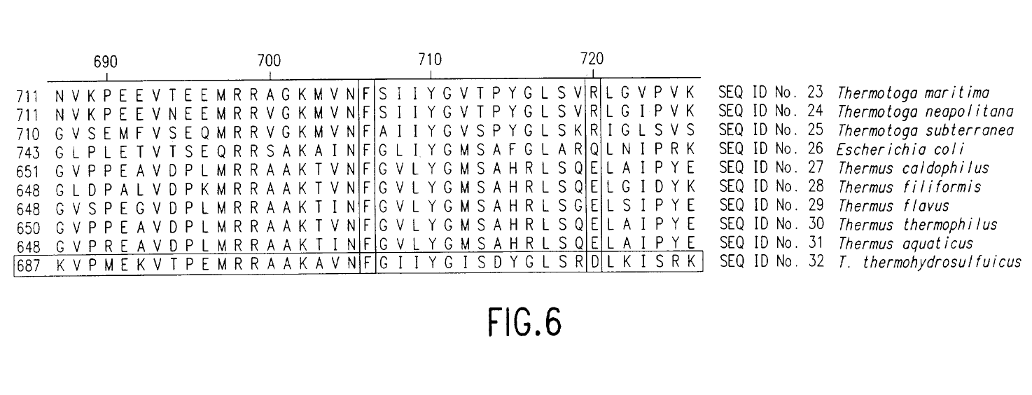

Alignment of Wild Type Pol I Sequences from Different Microorganisms FIG. 6.

Homologous positions in finger region of Polymerases of Pol I family are shown here. Note the alignment of amino acids corresponding to 720 of full-length (position 426 of ΔTts) Tts Pol I. The blocked region demonstrates the region of homology between the enzymes. The role of phenlyalanine at positions corresponding 706 of Tts Pol I in discrimination towards ddNTPs is shown for reference and has been documented in literature. The claims of this patent cover the role of amino acid at position 720 of full-length Tts DNA polymerase I. Alteration of this amino acid results in easy incorporation of dye-labeled nucleotide analogs. A negatively charged amino acid at this position is more discriminatory towards the incorporation of dye-labeled nucleotide. An alteration to positively charged residues such as arginine or lysine or other bulky residues results in the lowering of discrimination towards the dNTP or ddNTP conjugates. Besides the usefulness of other naturally occurring polymerases for dye nucleotide labeling, that naturally harbor residues other than glutamate or aspartate are also covered in this patent.

### Example 4

Assay Conditions

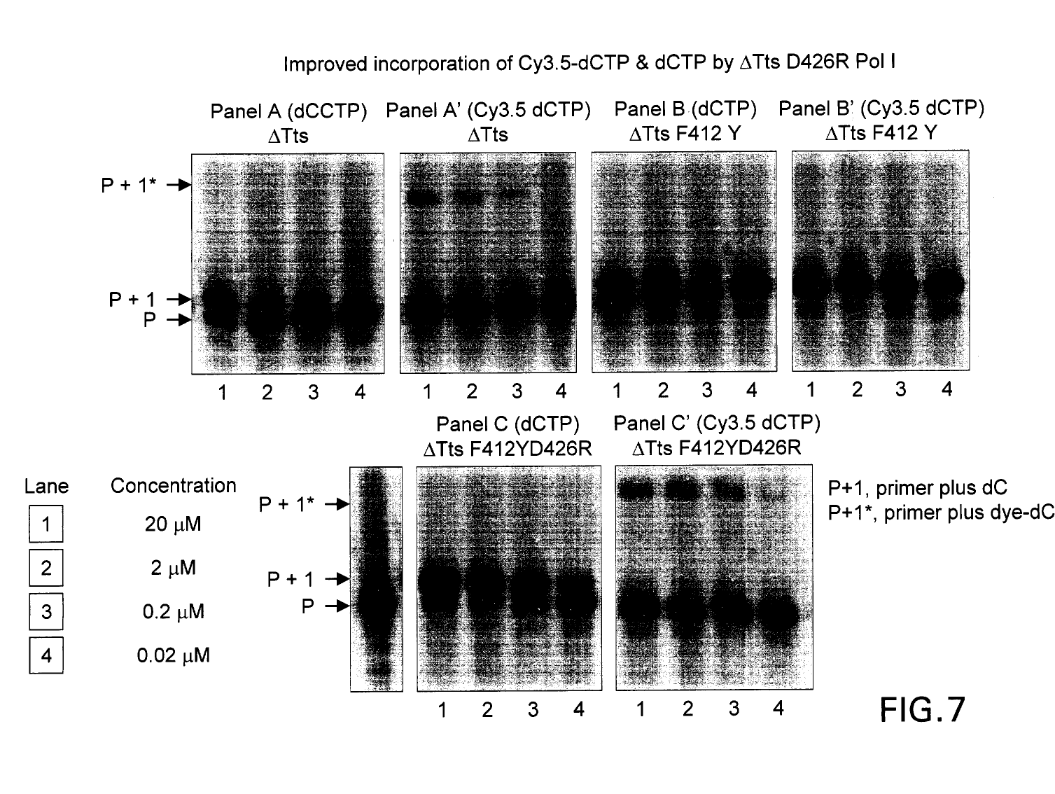

The experiment shown in FIG. 7 investigates the relative efficiencies of incorporation of dye-CTP (Cy3.5-dCTP) and dCTP. Three enzyme preparations ΔTts, ΔTts F412Y, ΔTtsF412YD426R were analyzed in this experiment.

Optimized 1×buffer compositions for Reverse Transcriptase (RNA dependent DNA Polymerase) and DNA Polymerase (DNA dependent DNA polymerase) reactions for all variants of Tts Pol I are as follows. Tris, pH 8.0 (50 mM), KCl (40 mM), MgCl2 (3 mM), DTT (1 mM), DNA or RNA template (as needed), primer (5 to 50 femto mols), enzyme 0.5 to 1 units, dNTP or dNTP analog (varying concentrations as needed). In standard synthesis reactions, when full-length synthesis is monitored, 50 uM of all 4 dNTPs are included. Typical reaction volume is 10 ul. Reaction temperature was kept between 37-60 C. depending on the experimental needs. Reaction time was limited to 10 minutes for single nucleotide incorporation studies. Time was varied as needed for the purpose of the experiments, sometimes up to 1 hour if longer extensions are monitored.

The experiment shown in FIG. 7 investigates the relative efficiencies of incorporation of dye-CTP (Cy3.5-dCTP) and dCTP. Three enzyme preparations ΔTts, ΔTtsFY, ΔTtsF412YD426R were analyzed in this experiment.

In FIG. 7, Globin mRNA served as the template. A 5′ P-33 labeled primer (DNA 25-mer) was annealed to the template. Reactions were performed with varying concentrations of either Cy3.5-dCTP or dCTP alone. Inclusion of only one dNTP allowed incorporation of the next correct nucleotide alone. The sequence of the template-primer that allowed for the examination of single nucleotide “C” incorporation is shown below.

| | | |
|---|---|---|
| CAGGCTGCCTATCAGAAGGTGGTGGCTGGTGTGGCCkATGCCCTGGCTCA | 5′ 3′ mRNA | (SEQ ID No. 11) |
| TTCCACCACCGACCACACCGGTTACGG* | CP-16 3′-5′ | (SEQ ID No. 12) |

Lanes 1,2,3 and 4 contain 20, 2, 0.2, and 0.02 uM of dNTP or dye-dNTP. Lane with no label has no enzyme, to show the integrity of the starting P-33 labeled primer. Quantification of the single nucleotide extension product is one way to tell if DR change to in the enzyme led to any consequence. “P” indicates a radio labeled primer. “P+1” represents the elongated product by a single nucleotide or nucleotide analog. P+1 migrates slowly with the dye-dNTP conjugate. Note that the migration of Cy3.5 dCMP containing bands travel slowly on the gel compared to dCTP extended products. Comparing Panel A, B and C, lanes 1 through 4 shows that the alteration of the amino acid back bone of the enzyme from D to R results in the improved efficiency of natural nucleotides. P+1 product is achieved between 10-100 fold less concentration of dCTP with the enzyme having the DR change Likewise, a comparison of panels A′, B′ and C′ reveals that the alteration DR results in improved incorporation of dye-CTP. Essentially, incorporation is achieved at lower concentrations (less than 10 times) of dye-dNTP compared to that of the wild type polymerase. It is evident that the DR enzyme was able to incorporate Cy3.5 dCTP at concentrations as low as 0.2 uM (Cy3.5 dCTP) or even lower. Compare this with the D enzyme, which exhibits relatively poor incorporation at these lower concentrations. And the results also show that this mutation dramatically reverses the decreased incorporation of dye-CTP seen with Tts F412Y in panel B′. In this experiment the template-primer is a mRNA annealed to radio labeled primer. Extension is monitored qualitatively as P+1 for the natural nucleotides and P+1* for dye labeled nucleotide. This is a promising first observation for potential use in microarray applications for cDNA probe labeling.

### Example 5

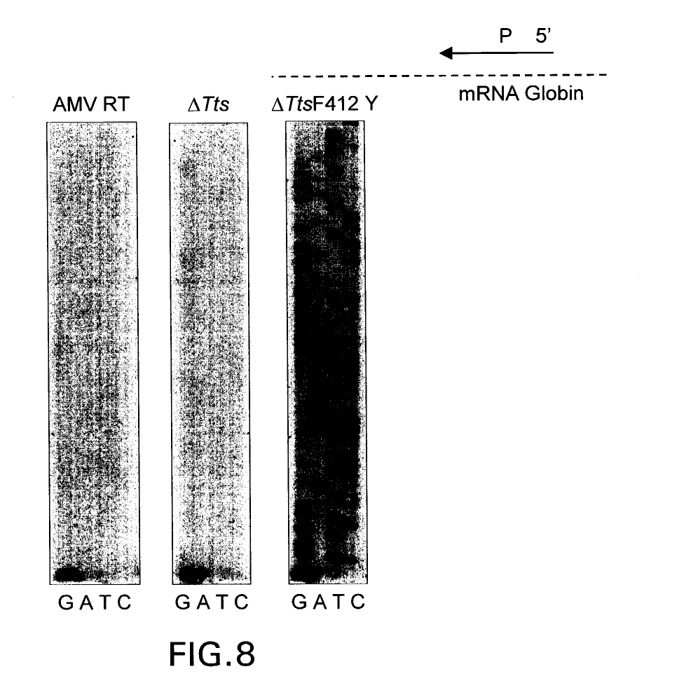

Direct RNA Sequencing by AMV RT, ΔTts and ΔTtsF412Y Pol I (FIG. 8).

Globin mRNA served as a template. The 50-mer DNA was used a primer. Standard sequencing components in Amersham Pharmacia Thermo Sequenase kit were employed for sequencing. P-33 labeled terminators (ddNTP) were obtained from Amersham Pharmacia Biotech. Post-sequencing reaction products were separated on 6% urea-polyacrylamide gels. AMV, Avian Myeloblastosis virus RT.

### Example 6

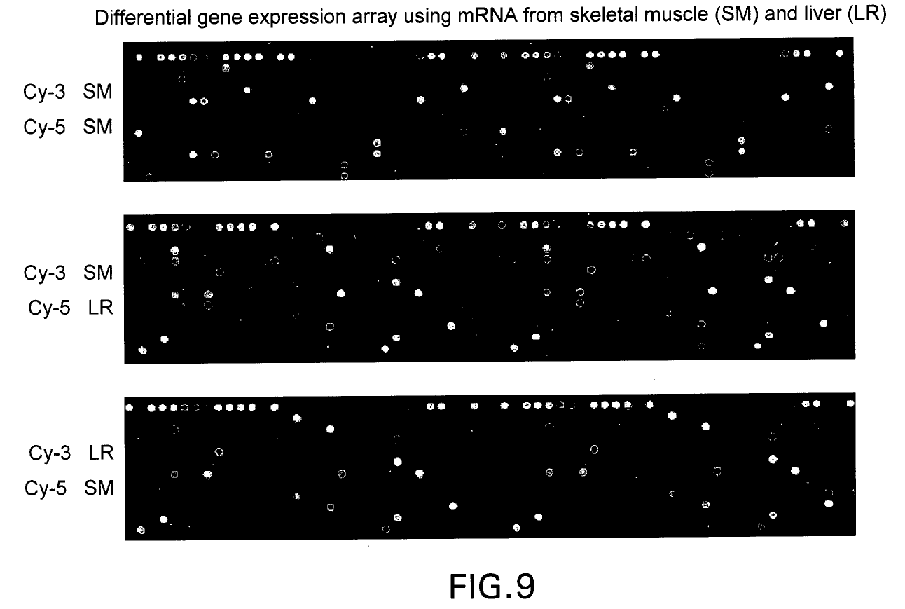

ΔTtsF412YD426R Performance in cDNA Labeling Using Cy3 and Cy5-dCTP and Utility in Microarray Applications (FIG. 9).

A typical 20 μl reaction Cy3 or Cy5 reaction had 1 μg of human skeletal muscle mRNA, oligo dT₍₂₅₎ and random nonamer primers and TtsFYDR polymerase enzyme in 1×reaction buffer (50 mM Tris, pH 8.0, 1 mM DDT, 40 mM KC, 100 uM dA,G and TTP and 50 um each of CTP and Cy3-dCTP or Cy5-dCTP depending on the reaction). Control mRNAs (APBiotech Inc.) of known sequence compositions were included in various concentrations to serve as dynamic range and gene expression ratio controls. Tts reactions were carried out at temperatures from 50 degrees C. Template RNA was hydrolyzed by alkali treatment and neutralized with HEPES.

Probes were purified using MultiScreen filters (Millipore) and quantified by spectrophotometry. Glass slides containing human cDNA gene targets were hybridized with equal amounts (30 pmol each) of Cy3 and Cy5 labeled cDNA probes. Slides were scanned using a GenePix® (Axon) scanner and quantified using ImageQuant software. The figure illustrates the accurate representation of probes, near even incorporation of Cy3 and Cy5 and differential gene expression in Cy3 versus Cy5 reactions.

### Example 7

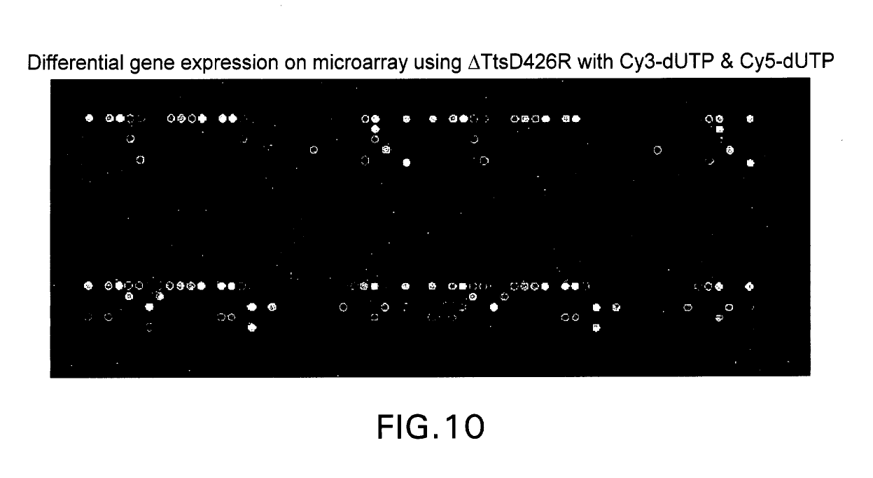

ΔTtsF412YD426R Performance in cDNA Labeling Using Cy3 and Cy5-dUTP and Utility in Microarray Applications (FIG. 10).

A typical 20 μl reaction Cy3 or Cy5 reaction had 1 μg of human skeletal muscle mRNA, oligo dT₍₂₅₎ and random nonamer primers and TtsFYDR polymerase enzyme in 1×reaction buffer (50 mM Tris, pH 8.0, 1 mM DDT, 40 mM KC, 100 uM dA,G and CTP and 50 um each of TTP and Cy3-dUTP or Cy5-dUTP depending on the reaction). Control mRNAs (APBiotech Inc.) of known sequence compositions were included in various concentrations to serve as dynamic range and gene expression ratio controls. Tts reactions were carried out at temperatures from 50 degrees C. Template RNA was hydrolyzed by alkali treatment and neutralized with HEPES.

Probes were purified using MultiScreen filters (Millipore) and quantified by spectrophotometry. Glass slides containing human cDNA gene targets were hybridized with equal amounts (30 pmol each) of Cy3 and Cy5 labeled cDNA probes. Slides were scanned using a GenePix® (Axon) scanner and quantified using ImageQuant software. The figure illustrates the accurate representation of probes, near even incorporation of Cy3 and Cy5 and differential gene expression in Cy3 versus Cy5 reactions.

### Example 8

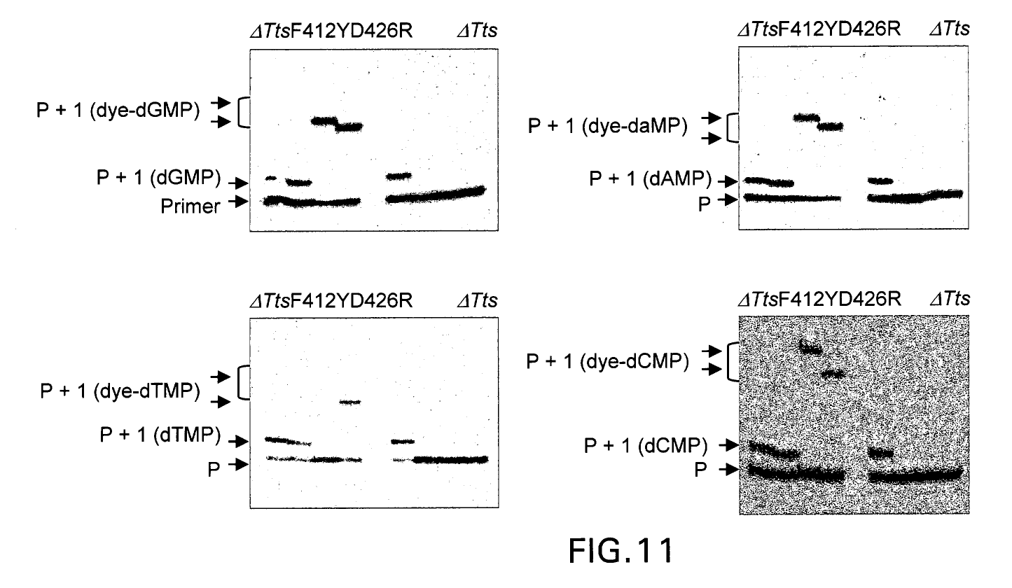

Usefulness of ΔTtsF412Y or ΔTtsF412YD426R Pol I in SnuPE, single nucleotide primer extension for investigation of target base on RNA (FIG. 11). Lane 1 is dNTP (G, A, T or C in panels A, B, C and D). Lane 2 is cold ddNTP. Lane 3 is a dye labeled ddNTP (linker arm length eleven carbon atoms). Lane 4 is a dye labeled ddNTP (linker arm length four carbon atoms). The dyes are from rhodamine class of FAM, R6G, TMR and Rox conjugated to ddG, ddA, ddT and ddC by either a 4-carbon or 11-carbon linkage.

The sequence of the template-primer that allowed for the examination of single nucleotide “G” incorporation is shown below.

| | | |
|---|---|---|
| CAGGCTGCCTATCAGAAGGTGGTGGCTGGTGTGGCCAATGCCCTGGCTCA | 5′ 3′ mRNA | (SEQ ID No. 13) |
| TCTTCCACCACCGACCACACCGGTTACGG* | CP-18 (3′-5′) | (SEQ ID No. 14) |

The sequence of the template-primer that allowed for the examination of single nucleotide “A” incorporation is shown below.

| | | |
|---|---|---|
| CAGGCTGCCTATCAGAAGGTGGTGGCTGGTGTGGCCAATGCCCTGGCTCA | 5′ 3′ mRNA | (SEQ ID No. 15) |
| GTCTTCCACCACCGACCACACCGGTTACGG* | CP-19 (3′-5′) | (SEQ ID No. 16) |

The sequence of the template-primer that allowed for the examination of single nucleotide “T” incorporation is shown below.

| | | |
|---|---|---|
| CAGGCTGCCTATCAGAAGGTGGTGGCTGGTGTGGCCAATGCCCTGGCTCA | 5′ 3′ mRNA | (SEQ ID No. 17) |
| CTTCCACCACCGACCACACCGGTTACGG* | CP-17 (3′-5′) | (SEQ ID No. 18) |

The sequence of the template-primer that allowed for the examination of single nucleotide “C” incorporation is shown below.

| | | |
|---|---|---|
| CAGGCTGCCTATCAGAAGGTGGTGGCTGGTGTGGCCAATGCCCTGGCTCA | 5′ 3′ mRNA | (SEQ TD No. 19) |
| TTCCACCACCGACCACACCGGTTACGG* | CP-16 (3′-5′) | (SEQ ID No. 20) |

Assays for measuring efficiency of dye-ddNTP or ddNTP by Tts variants were measured as below. A cocktail containing all reaction components except the ddNTP or dye-ddNTP was prepared as below. The reactions contained the following components. Tris, pH 8.0 (50 mM), KCl (40 mM), MgCl₂, (3 mM) DTT (1 mM) dNTP or dye dNTP 0.2 uM (lanes 1, 2, 3 and 4), 5′ labeled p-33 primer (0.2 pimol), mRNA globin Template (100 ng), enzyme in a 10-ul reaction volume.

Template-primer annealing was accomplished by treating the components at 60 C. for 10 minutes followed by slowly cooling to 37 C. to allow for proper annealing. Reactions carried out for 10 min at appropriate temperature. Reactions were terminated by addition of 6 ul of formamide-stop solution. Samples were separated and analyzed on a 16% denaturing polyacrylamide gel. The wet gel was dried on Whatmann Filter paper and imaged using Autoradiography or PhosPhor Imager.

### Example 9

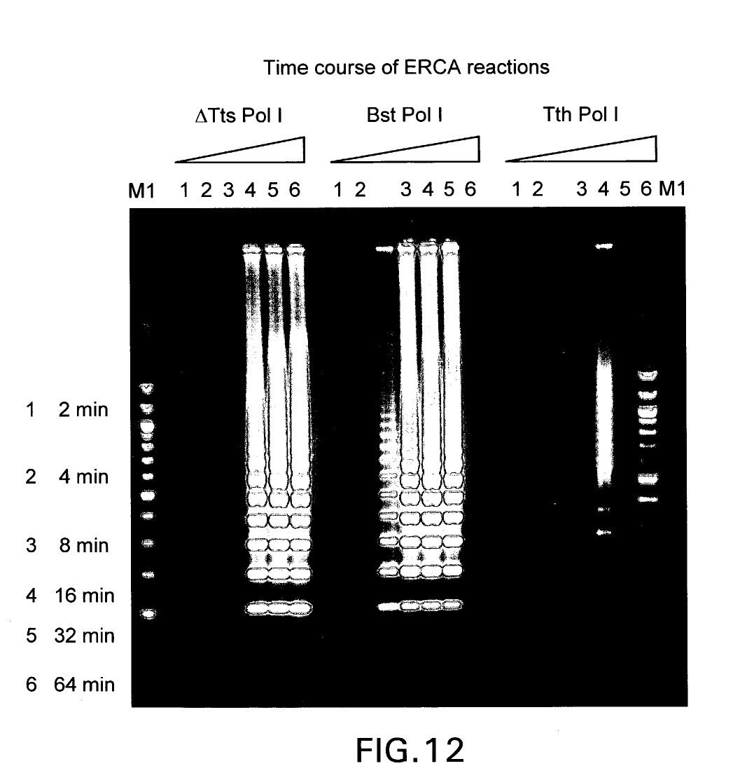

Utility of Either ΔTts in RCA Reaction (FIG. 12)

Isothermal Rolling circle amplification reactions were performed as below. Template circular DNA was with primers 1 (Complementary to the circle) and 2 (same polarity as the circle), with all the components including the enzyme were combined as below. The reactions were performed at 55 C. for an hour and products analyzed following separation on 1% agarose gel. A 20-ul reaction contained, Tris pH 8.0 (50 uM), KCl (40 uM), MgCl2 (3 uM), DTT (1 uM) and dNTP (400 uM), primer 1 & 2 (1 uM, each), template and enzyme 20 units. Tth pol I reactions were done at 70 C. and Bst DNA pol reactions carried out at 55 C.

### Example 10

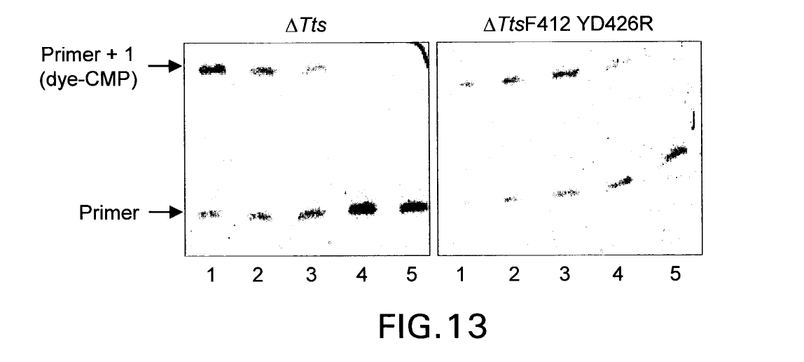

Incorporation of Dye Labeled Nucleotide During DNA Dependent DNA Synthesis ΔTts, ΔTtsF412Y, ΔTtsF412YD426R (FIG. 13).

Primer extension was monitored using a defined DNA template-DNA primer.

In this experiment the relative efficiencies of incorporation of dye-CTP (Cy3.5-dCTP) was investigated. Three enzyme preparations were tested ΔTts, ΔTtsF412Y, and ΔTtsF412YD426R

Defined DNA shown below served as the template. A P-33 labeled primer (DNA 25 mer) was annealed to the template. Reactions were performed with varying concentrations of either Cy3.5-dCTP or dCTP alone. Inclusion of only one dNTP allowed incorporation of the next correct nucleotide alone. The sequence of the template-primer that allowed for the examination of single nucleotide “C” incorporation is shown below.

| | | |
|---|---|---|
| CAGGCTGCCTATCAGAAGGTGGTGGCTGGTGTGGCCAATGCCCTGGCTCA | 5′ 3′ DNA | (SEQ ID No. 21) |
| TTCCACCACCGACCACACCGGTTACGG* | CP-16 3′-5′ | (SEQ hID No. 22) |

Lanes 1, 2, 3, 4 and 5 contain 20, 2, 0.2, 0.02 and 0 uM dye-dCTP. Lane 5 has no enzyme, to show the integrity of the starting p-33 labeled primer. Quantification of the single nucleotide extension product is one way to tell if DR change in the enzyme led to any consequence. “P” indicates a radio labeled primer. “P+1” represents the elongated product by a single nucleotide or nucleotide analog. P+1 migrates slowly with the dye-dNTP conjugate. Note that the migration of Cy3.5-dCTP containing band travel slowly on the gel compared to dCMP extended products. Comparing Panel A, B and C, lanes 1 through 4 shows that the alteration of the amino acid back bone of the enzyme from D to R results in the improved efficiency of natural nucleotides. P+1 product is achieved between 10-100 fold less concentration of dye-dCTP with the enzyme having the DR change. Essentially, incorporation is achieved at a much lower concentration of dye-dNMP compared to the wild type enzyme. It is evident that the DR enzyme was able to incorporate Cy3.5-dCTP at concentrations as low as 0.2 uM or even lower. Compare this with the D enzyme (wild type), which exhibits relatively poor incorporation at these lower concentrations. And the results also show that this mutation dramatically reverses the decreased incorporation of dye-CMP seen with Tts F412Y in panel B. These observations demonstrate the utility of a DR variant in labeling during DNA template dependent synthesis as well.

### Example 11

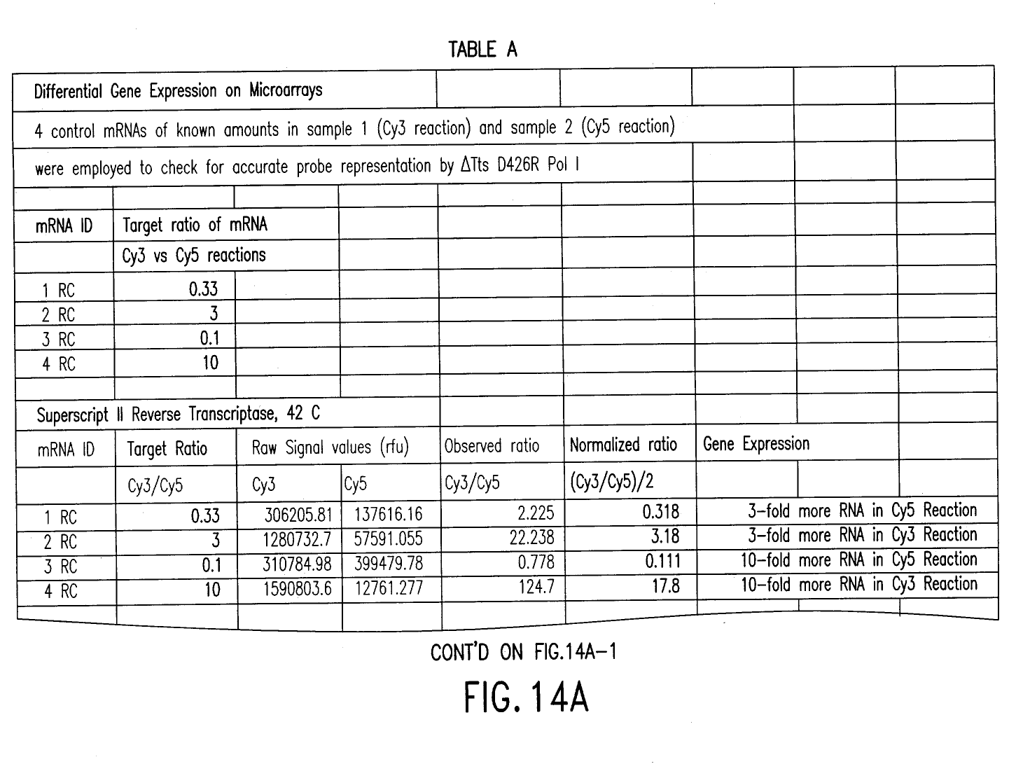

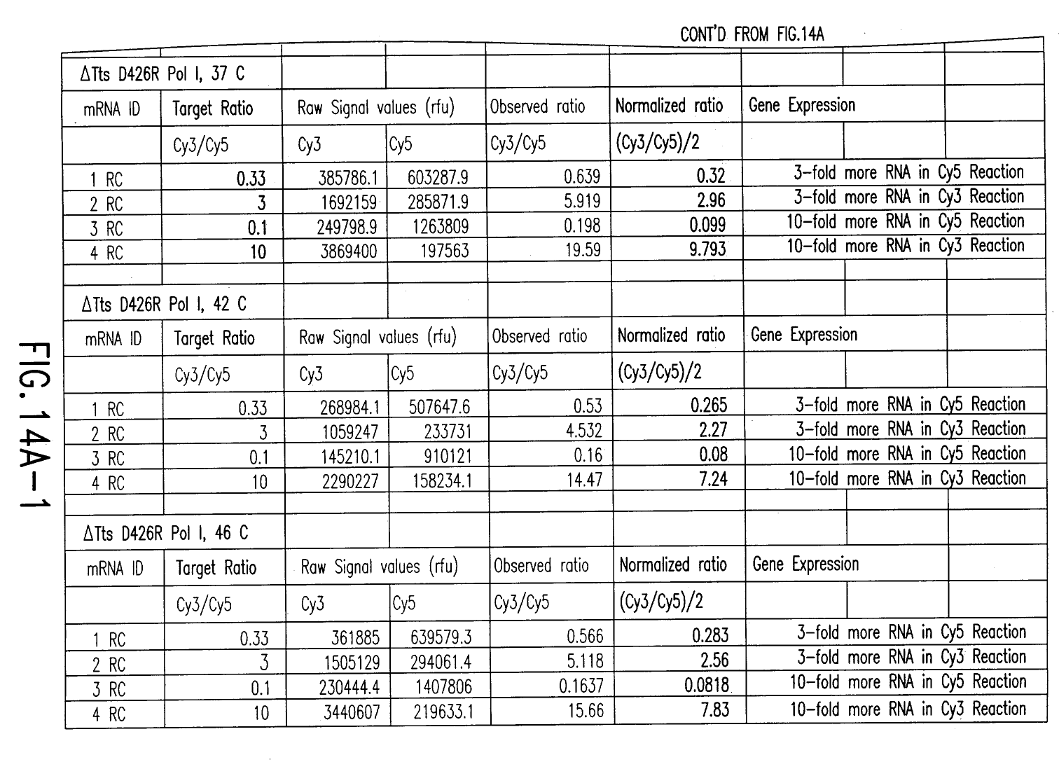

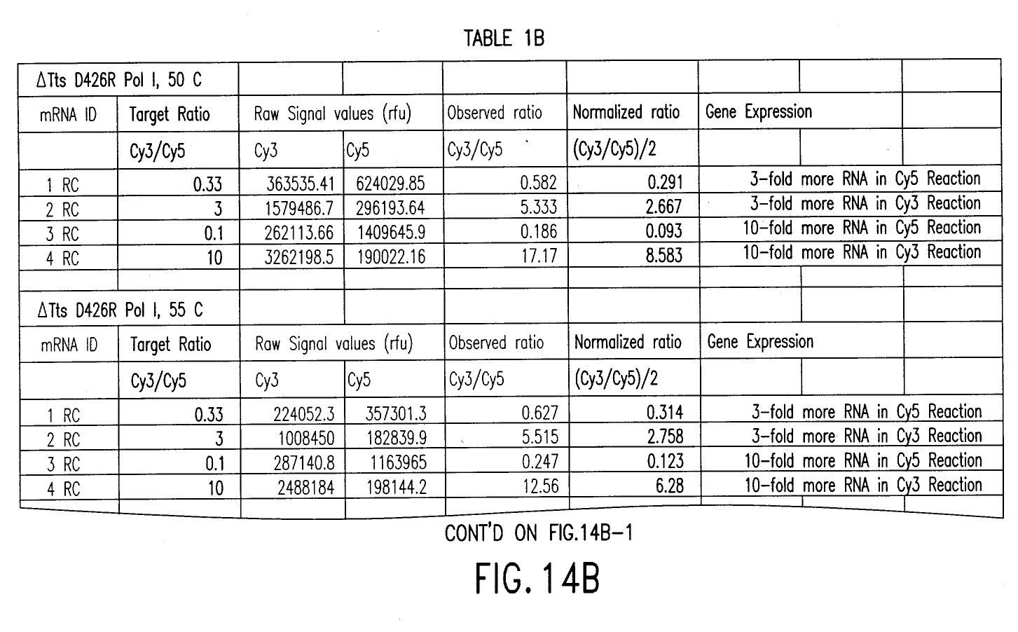

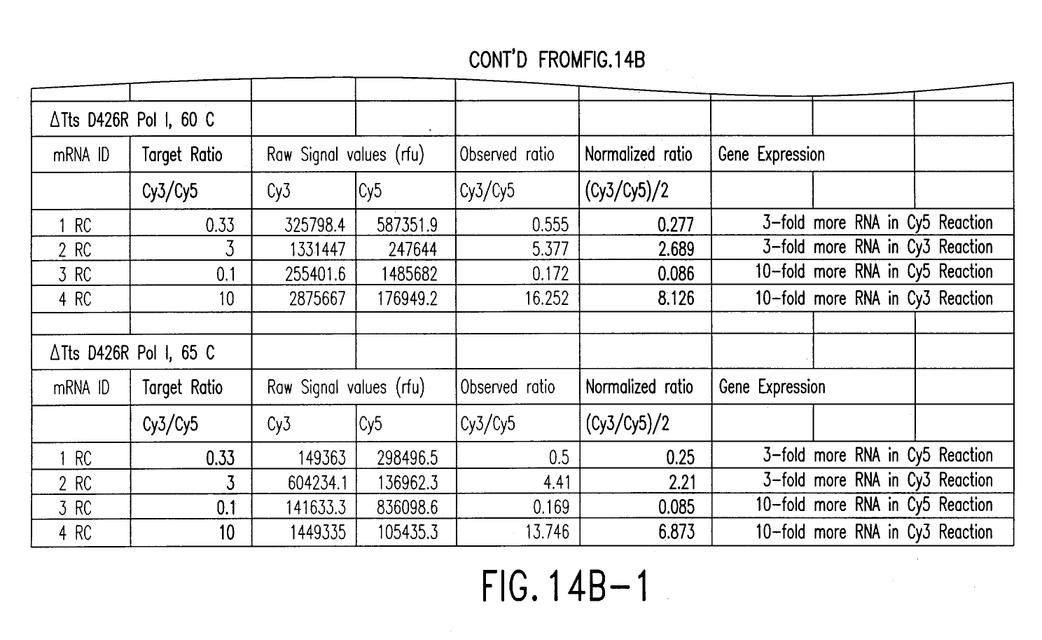

ΔTtsF412YD426R Performance in cDNA Labeling Using Cy3/Cy5-dCTP; Demonstration of Accurate Determination of Gene Expression Over a Wide Reaction Temperature Range (FIGS. 14a and b).

A 20 μl reaction Cy3 or Cy5 reaction had 1 μg of human skeletal muscle mRNA, oligo dT₍₂₅₎ and random nonamer primers and TtsFYDR polymerase enzyme in 1×reaction buffer (50 mM Tris, pH 8.0, 1 mM DDT, 40 mM KC, 100 uM dA,G and TTP and 50 um each of CTP and Cy3-dCTP or Cy5-dCTP depending on the reaction). Control mRNAs (APBiotech Inc.) of known sequence compositions were included in various concentrations to serve as dynamic range and gene expression ratio controls. Tts reactions were carried out at temperatures from 37, 42, 45, 50, 55, 60 and 65 degrees. For Superscript II, cDNA synthesis reactions were carried out at 42 C. (Life Technologies). Template RNA was hydrolyzed by alkali treatment and neutralized with HEPES.

Probes were purified using MultiScreen filters (Millipore) and quantified by spectrophotometry. Glass slides containing human cDNA gene targets were hybridized with equal amounts (30 pmol each) of Cy3 and Cy5 labeled cDNA probes. Slides were scanned using a GenePix® (Axon) scanner and quantified using ImageQuant software. A normalization factor of 2 (due to differences in the excitation efficiencies of Cy3 and Cy5) was applied to the observed ratio of raw flourescence signal. The figure illustrates precise determination of gene expression differences in Cy3 and Cy5 reactions. For example across all temperature ranges the normalized observed ratios were very close to the target ratios demonstrating the ability to accurately determine gene expression differences over a wide temperature range using ΔTtsF412YD426R.

### Example 12

Protein Purification Protocol ΔTtsF412YD426R or ΔTtsD426R pol I Purification Scheme

The following was the protocol adapted for cells harvested from 1 L of LB media for initial enzyme evaluation studies (Typical yield of wet cells 4-6 g). E. coli cells harboring the expression vector were grown according to standard protocols as described in the original patent and harvested and kept frozen until ready for use. Cell lysis was carried out by adding 5 ml lysis buffer for every gram of wet cell paste (50 mM Tris pH 8.0, 1 mM EDTA, 50 mM NaCl, 10% Glycerol and containing 1 mg/ml lysozyme). Cells were left on ice for 40 minutes. Upon complete resuspension the cells were passed through a French Press at 15,000 PSI. After lysis, cell extract was treated at 70 C. for 10 to inactivate host enzymes. The extract was then clarified following centrifugation at 12,000 rpm for 30 minutes. The supernatant containing the enzyme fraction was then used for further purification.

The lysate was then loaded on to a Q-Sepharose HP column previously equilibrated with buffer A (Tris 50 mM (pH 7.5), EDTA 1 mM, NaCl 150 mM, 10% glycerol). The column was washed four times with buffer A. The flow rate of the buffer was 8- 10 ml per minute. This step selectively binds nucleic acid and the follow-through containing the enzyme is used in subsequent column. The flow-through sample was concentrated to small volume and removed of salt by tangential flow and diafiltration device to prepare for the next column. The sample was loaded on to a second Q-Sepharose HP column pre-equilibrated with buffer B (Tris 50 mM (pH 7.5), EDTA 1 mM, 10% glycerol). The column was washed with buffer B for three additional column volumes to remove any unbound proteins. The ΔTts F412YD426R pol I preparation was eluted by establishing a 0-30% gradient salt using NaCl. The eluted sample was dialyzed against buffer C (30 mM sodium phosphate, 30 mM sodium formate, 60 mM sodium acetate, 1 mM EDTA and 10% glycerol). The dialyzed sample was loaded on to a Resource S column previously equilibrated with buffer C. Column was washed with buffer C for three additional column volumes to remove unbound proteins. ΔTtsF412YD426R Pol I was eluted specifically using a 0-50% salt gradient using NaCl. This sample contained the purified enzyme preparation.

A similar purification protocol was employed for ΔTtsD426R Pol I.

What is claimed is:

1. An enzymatically active DNA polymerase or active fragment thereof, having at least 80% identity in its amino acid sequence to the DNA polymerase of Thermoanaerobacter thermohydrosulfuricus or a fragment thereof, and having an amino acid alteration at position 720 in Tts Pol I or at position 426 in ΔTts Pol I or at a homologous position defined with respect to Tts DNA ploymerase I, having improved incorporation of nucleotide analogs and natural bases during DNA synthesis compared to unaltered enzyme.

2. The polymerase of claim 1 wherein said nucleotide analogs are dNTP, ddNTP and rNTP analogs.

3. The polymerase of claim 2 wherein said dNTP, ddNTP and rNTP analogs are dye-conjugated or biotin conjugated dNTP, ddNTP and rNTP.

4. The polymerase of claim 3 wherein the dye in said dye-conjugated dNTP, ddNTP and rNTP is a rhodamine or Cyanine derivative dye.

5. The polymerase of claim 4 wherein said rhodamine dye is R110, R6G, TMR or Rox.

6. The polymerase of claim 4 wherein said Cyanine derivative dye is Cy3, Cy3.5, Cy5.0 or Cy5.5.

7. The polymerase of claim 1, wherein said polymerase has the aspartate at position 720 in Tts Pol I or at position 426 in ΔTts Pol I, replaced with arginine.

8. Method of performing direct RNA sequencing utilizing the polymerase of claim 1.

9. Method of incorporating Cy3 and Cy5 dye conjugated dNTPs across a range of reaction temperatures from 37-65 C. utilizing the polymerase of claim 1.

10. Kit for labeling a polynucleotide from a DNA or RNA template with a DNA or RNA primer comprising a DNA polymerase of claim 1.


SEQUENCE LISTING (32 sequences)

SEQ ID NO: 1 - protein, 872 aa, Thermoanaerobacter thermohydrosulfuricus
MYKFLIIDGSSLMYRAYYALPMLTTSEGLPTNALYGFTMMLIKLIEEEKPDYIAIAFDKKAPTFRHKEYQDYKATRQAMPEELAEQVDYLKEIIDGFNIKTLELEGYEADDIIGTISKLAEEKGMEVLVVTGDRDALQLVSDKVKIKISKKGITQMEEFDEKAILERYGITPQQFIDLKGLMGDKSDNIPGVPNIGEKTAIKLLKDFGTIENLIQNLSQLKGKIKENIENNKELAIMSKRLATIKRDIPIEIDFEEYKVKKFNEEKLLELFNKLEFFSLIDNIKKESSIEIVDNHKVEKWSKVDIKELVTLLQDNRNIAFYPLIYEGEIKKIAFSFGKDTVYIDVFQTEDLKEIFEKEDFEFTTHEIKDFLVRLSYKGIECKSKYIDTAVMAYLLNPSESNYDLDRVLKKYLKVDVPSYEGIFGKGRDKKKIEEIDENILADYICSRCVYLFDLKEKLMNFIEEMDMKKLLLEIEMPLVEVLKSMEVSGFTLDKEVLKELSQKIDDRIGEILDKIYKEAGYQFNVNSPKQLSEFLFEKLNLPVIKKTKTGYSTDSEVLEQLVPYNDIVSDIIEYRQLTKLKSTYIDGFLPLMDENNRVHSNFKQMVTATGRISSTEPNLQNIPIREEFGRQIRRAFIPRSRDGYIVSADYSQIELRVLAHVSGDEKLIESFMNNEDIHLRTASEVFKVPMEKVTPEMRRAAKAVNFGIIYGISDYGLSRDLKISRKEAKEYINNYFERYKGVKDYIEKIVRFAKENGYVTTIMNRRRYIPEINSRNFTQRSQAERLAMNAPIQGSAADIIKMAMVKVYNDLKKLKLKSKLILQVHDELVVDTYKDEVDIIKKILKENMENVVQLKVPLVVEIGVGPNWFLAK

SEQ ID NO: 2 - DNA, 2619 bp, Thermoanaerobacter thermohydrosulfuricus
atgtataaatttttaataattgatggaagtagcctcatgtacagagcctattatgccttgcccatgcttactacaagtgagggattgcctacaaatgctctgtatggttttactatgatgcttataaaacttatcgaggaggaaaaacctgattacatagctattgcttttgacaaaaaagctcctacttttagacacaaagaatatcaagactacaaagctacaagacaagctatgcctgaagaacttgctgaacaagtagactatttgaaagaaattatagatggctttaatataaagacattagaattagaaggttatgaagctgatgacattatagggactatttcaaagctggcagaggaaaaaggaatggaagtgcttgtagttacaggagacagagatgctcttcaattagtttcagataaagtgaagataaaaatttctaaaaagggtattactcagatggaagagtttgacgaaaaggctattttagaaaggtatggaataactcctcagcagtttatagatttaaaagggcttatgggagataaatctgataatatccctggagtacctaatataggggaaaaaactgcgattaagctattaaaggattttggaacaattgaaaatttaatccaaaatctttctcagcttaaaggtaaaataaaagaaaatatagaaaacaataaagagttagctataatgagtaagaggcttgctactataaaaagagacattcccattgagatagattttgaggagtataaagtaaaaaaatttaatgaggagaagcttttagagctttttaataaattagaattctttagtttaattgataacataaagaaagaaagtagcatagagattgtagataatcataaagttgaaaaatggtcaaaagtagatataaaagaattagtaactttgttgcaagataacagaaatattgctttttacccgttaatttatgaaggggaaataaaaaaaatagccttttcttttggaaaggatacggtttatattgacgttttccaaacagaagatttaaaggagatttttgaaaaagaagattttgaatttacaacccatgaaataaaggattttttagtgaggctttcttataaaggaatagagtgtaaaagcaagtacatagatactgctgtaatggcttatcttctgaatccttctgagtctaactatgacttagaccgtgtgctaaaaaaatatttaaaggtagatgtgccttcttatgaaggaatatttggcaaaggtagggataaaaagaaaattgaagagattgacgaaaacatacttgctgattatatttgcagtagatgtgtgtatctatttgatttaaaagaaaagctgatgaattttattgaagagatggatatgaaaaaacttctattagaaatagaaatgcctcttgtagaagttttaaaatcaatggaggtaagtggttttacattggataaagaagttctaaaagagctttcacaaaagatagatgatagaataggagaaatactagataaaatttataaagaggcaggatatcaatttaatgtaaattcacctaagcaattaagtgaatttttgtttgaaaagttaaacttaccagtaataaagaaaacaaaaacaggatactctacggattctgaagttttggaacaattggttccttataatgatattgtcagcgatataatagagtatcggcaacttacaaaacttaaatctacttatatagatggatttttgcctcttatggatgaaaacaatagagtacattctaattttaaacaaatggttactgctacaggtagaataagcagcaccgagccaaatctacaaaatatacctataagagaagagtttggcagacaaattagaagggcttttattccgaggagtagagatggatatattgtttcagcagattattctcagattgaactgagggttttagcacatgtttcgggagatgaaaagctaatagaatcttttatgaataatgaagatatacatttaaggacagcttcggaggtttttaaagttcctatggaaaaagttacaccggagatgagaagagcagcaaaagccgtaaattttggcataatatatggcataagcgattatgggctttctcgagaccttaaaatatcaagaaaagaagcaaaagagtacataaataattattttgaaagatataaaggagtaaaagattatattgaaaaaatagtacgatttgcaaaagaaaatggctatgtgactacaataatgaacagaaggagatatattcctgaaataaactcaagaaattttactcaaagatcgcaggccgaaaggttagcaatgaatgctccgatacagggaagtgcggctgatataataaaaatggcaatggttaaggtatacaacgatttaaaaaaattaaagcttaagtctaagcttatattgcaagttcatgacgagcttgtagtggatacttataaggatgaagtagatatcataaaaaagatacttaaagaaaatatggaaaatgtagtgcaattaaaagttcctctggttgttgaaattggcgtagggcctaattggtttttggccaagtga

SEQ ID NO: 3 - protein, 872 aa, Thermoanaerobacter thermohydrosulfuricus; identical to SEQ ID NO: 1

SEQ ID NO: 4 - DNA, 1737 bp, Thermoanaerobacter thermohydrosulfuricus
atgaaagttgaaaaatggtcaaaagtagatataaaagaattagtaactttgttgcaagataacagaaatattgctttttacccgttaatttatgaaggggaaataaaaaaaatagccttttcttttggaaaggatacggtttatattgacgttttccaaacagaagatttaaaggagatttttgaaaaagaagattttgaatttacaacccatgaaataaaggattttttagtgaggctttcttataaaggaatagagtgtaaaagcaagtacatagatactgctgtaatggcttatcttctgaatccttctgagtctaactatgacttagaccgtgtgctaaaaaaatatttaaaggtagatgtgccttcttatgaaggaatatttggcaaaggtagggataaaaagaaaattgaagagattgacgaaaacatacttgctgattatatttgcagtagatgtgtgtatctatttgatttaaaagaaaagctgatgaattttattgaagagatggatatgaaaaaacttctattagaaatagaaatgcctcttgtagaagttttaaaatcaatggaggtaagtggttttacattggataaagaagttctaaaagagctttcacaaaagatagatgatagaataggagaaatactagataaaatttataaagaggcaggatatcaatttaatgtaaattcacctaagcaattaagtgaatttttgtttgaaaagttaaacttaccagtaataaagaaaacaaaaacaggatactctacggattctgaagttttggaacaattggttccttataatgatattgtcagcgatataatagagtatcggcaacttacaaaacttaaatctacttatatagatggatttttgcctcttatggatgaaaacaatagagtacattctaattttaaacaaatggttactgctacaggtagaataagcagcaccgagccaaatctacaaaatatacctataagagaagagtttggcagacaaattagaagggcttttattccgaggagtagagatggatatattgtttcagcagattattctcagattgaactgagggttttagcacatgtttcgggagatgaaaagctaatagaatcttttatgaataatgaagatatacatttaaggacagcttcggaggtttttaaagttcctatggaaaaagttacaccggagatgagaagagcagcaaaagccgtaaattttggcataatatatggcataagcgattatgggctttctcgagaccttaaaatatcaagaaaagaagcaaaagagtacataaataattattttgaaagatataaaggagtaaaagattatattgaaaaaatagtacgatttgcaaaagaaaatggctatgtgactacaataatgaacagaaggagatatattcctgaaataaactcaagaaattttactcaaagatcgcaggccgaaaggttagcaatgaatgctccgatacagggaagtgcggctgatataataaaaatggcaatggttaaggtatacaacgatttaaaaaaattaaagcttaagtctaagcttatattgcaagttcatgacgagcttgtagtggatacttataaggatgaagtagatatcataaaaaagatacttaaagaaaatatggaaaatgtagtgcaattaaaagttcctctggttgttgaaattggcgtagggcctaattggtttttggccaagtga

SEQ ID NO: 5 - protein, 872 aa, Thermoanaerobacter thermohydrosulfuricus
MYKFLIIDGSSLMYRAYYALPMLTTSEGLPTNALYGFTMMLIKLIEEEKPDYIAIAFDKKAPTFRHKEYQDYKATRQAMPEELAEQVDYLKEIIDGFNIKTLELEGYEADDIIGTISKLAEEKGMEVLVVTGDRDALQLVSDKVKIKISKKGITQMEEFDEKAILERYGITPQQFIDLKGLMGDKSDNIPGVPNIGEKTAIKLLKDFGTIENLIQNLSQLKGKIKENIENNKELAIMSKRLATIKRDIPIEIDFEEYKVKKFNEEKLLELFNKLEFFSLIDNIKKESSIEIVDNHKVEKWSKVDIKELVTLLQDNRNIAFYPLIYEGEIKKIAFSFGKDTVYIDVFQTEDLKEIFEKEDFEFTTHEIKDFLVRLSYKGIECKSKYIDTAVMAYLLNPSESNYDLDRVLKKYLKVDVPSYEGIFGKGRDKKKIEEIDENILADYICSRCVYLFDLKEKLMNFIEEMDMKKLLLEIEMPLVEVLKSMEVSGFTLDKEVLKELSQKIDDRIGEILDKIYKEAGYQFNVNSPKQLSEFLFEKLNLPVIKKTKTGYSTDSEVLEQLVPYNDIVSDIIEYRQLTKLKSTYIDGFLPLMDENNRVHSNFKQMVTATGRISSTEPNLQNIPIREEFGRQIRRAFIPRSRDGYIVSADYSQIELRVLAHVSGDEKLIESFMNNEDIHLRTASEVFKVPMEKVTPEMRRAAKAVNYGIIYGISDYGLSRDLKISRKEAKEYINNYFERYKGVKDYIEKIVRFAKENGYVTTIMNRRRYIPEINSRNFTQRSQAERLAMNAPIQGSAADIIKMAMVKVYNDLKKLKLKSKLILQVHDELVVDTYKDEVDIIKKILKENMENVVQLKVPLVVEIGVGPNWFLAK

SEQ ID NO: 6 - DNA, 1737 bp, Thermoanaerobacter thermohydrosulfuricus
atgaaagttgaaaaatggtcaaaagtagatataaaagaattagtaactttgttgcaagataacagaaatattgctttttacccgttaatttatgaaggggaaataaaaaaaatagccttttcttttggaaaggatacggtttatattgacgttttccaaacagaagatttaaaggagatttttgaaaaagaagattttgaatttacaacccatgaaataaaggattttttagtgaggctttcttataaaggaatagagtgtaaaagcaagtacatagatactgctgtaatggcttatcttctgaatccttctgagtctaactatgacttagaccgtgtgctaaaaaaatatttaaaggtagatgtgccttcttatgaaggaatatttggcaaaggtagggataaaaagaaaattgaagagattgacgaaaacatacttgctgattatatttgcagtagatgtgtgtatctatttgatttaaaagaaaagctgatgaattttattgaagagatggatatgaaaaaacttctattagaaatagaaatgcctcttgtagaagttttaaaatcaatggaggtaagtggttttacattggataaagaagttctaaaagagctttcacaaaagatagatgatagaataggagaaatactagataaaatttataaagaggcaggatatcaatttaatgtaaattcacctaagcaattaagtgaatttttgtttgaaaagttaaacttaccagtaataaagaaaacaaaaacaggatactctacggattctgaagttttggaacaattggttccttataatgatattgtcagcgatataatagagtatcggcaacttacaaaacttaaatctacttatatagatggatttttgcctcttatggatgaaaacaatagagtacattctaattttaaacaaatggttactgctacaggtagaataagcagcaccgagccaaatctacaaaatatacctataagagaagagtttggcagacaaattagaagggcttttattccgaggagtagagatggatatattgtttcagcagattattctcagattgaactgagggttttagcacatgtttcgggagatgaaaagctaatagaatcttttatgaataatgaagatatacatttaaggacagcttcggaggtttttaaagttcctatggaaaaagttacaccggagatgagaagagcagcaaaagccgtaaattatggcataatatatggcataagcgattatgggctttctcgagaccttaaaatatcaagaaaagaagcaaaagagtacataaataattattttgaaagatataaaggagtaaaagattatattgaaaaaatagtacgatttgcaaaagaaaatggctatgtgactacaataatgaacagaaggagatatattcctgaaataaactcaagaaattttactcaaagatcgcaggccgaaaggttagcaatgaatgctccgatacagggaagtgcggctgatataataaaaatggcaatggttaaggtatacaacgatttaaaaaaattaaagcttaagtctaagcttatattgcaagttcatgacgagcttgtagtggatacttataaggatgaagtagatatcataaaaaagatacttaaagaaaatatggaaaatgtagtgcaattaaaagttcctctggttgttgaaattggcgtagggcctaattggtttttggccaagtga

SEQ ID NO: 7 - protein, 872 aa, Thermoanaerobacter thermohydrosulfuricus
MYKFLIIDGSSLMYRAYYALPMLTTSEGLPTNALYGFTMMLIKLIEEEKPDYIAIAFDKKAPTFRHKEYQDYKATRQAMPEELAEQVDYLKEIIDGFNIKTLELEGYEADDIIGTISKLAEEKGMEVLVVTGDRDALQLVSDKVKIKISKKGITQMEEFDEKAILERYGITPQQFIDLKGLMGDKSDNIPGVPNIGEKTAIKLLKDFGTIENLIQNLSQLKGKIKENIENNKELAIMSKRLATIKRDIPIEIDFEEYKVKKFNEEKLLELFNKLEFFSLIDNIKKESSIEIVDNHKVEKWSKVDIKELVTLLQDNRNIAFYPLIYEGEIKKIAFSFGKDTVYIDVFQTEDLKEIFEKEDFEFTTHEIKDFLVRLSYKGIECKSKYIDTAVMAYLLNPSESNYDLDRVLKKYLKVDVPSYEGIFGKGRDKKKIEEIDENILADYICSRCVYLFDLKEKLMNFIEEMDMKKLLLEIEMPLVEVLKSMEVSGFTLDKEVLKELSQKIDDRIGEILDKIYKEAGYQFNVNSPKQLSEFLFEKLNLPVIKKTKTGYSTDSEVLEQLVPYNDIVSDIIEYRQLTKLKSTYIDGFLPLMDENNRVHSNFKQMVTATGRISSTEPNLQNIPIREEFGRQIRRAFIPRSRDGYIVSADYSQIELRVLAHVSGDEKLIESFMNNEDIHLRTASEVFKVPMEKVTPEMRRAAKAVNYGIIYGISDYGLSRRLKISRKEAKEYINNYFERYKGVKDYIEKIVRFAKENGYVTTIMNRRRYIPEINSRNFTQRSQAERLAMNAPIQGSAADIIKMAMVKVYNDLKKLKLKSKLILQVHDELVVDTYKDEVDIIKKILKENMENVVQLKVPLVVEIGVGPNWFLAK

SEQ ID NO: 8 - protein, 872 aa, Thermoanaerobacter thermohydrosulfuricus; identical to SEQ ID NO: 1

SEQ ID NO: 9 - DNA, 27 bp, Artificial Sequence
  Description of Artificial Sequence Primer
gggctttctcgacgccttaaaatatca

SEQ ID NO: 10 - DNA, 27 bp, Artificial Sequence
  Description of Artificial Sequence Primer
gccgtaaattttggcataatatatggc

SEQ ID NO: 11 - DNA, 50 bp, Thermoanaerobacter thermohydrosulfuricus
caggctgcctatcagaaggtggtggctggtgtggccaatgccctggctca

SEQ ID NO: 12 - DNA, 27 bp, Artificial Sequence
  Description of Artificial Sequence Primer
ggcattggccacaccagccaccacctt

SEQ ID NO: 13 - DNA, 50 bp, Thermoanaerobacter thermohydrosulfuricus; identical to SEQ ID NO: 11

SEQ ID NO: 14 - DNA, 29 bp, Artificial Sequence
  Description of Artificial Sequence Primer
ggcattggccacaccagccaccaccttct

SEQ ID NO: 15 - DNA, 50 bp, Thermoanaerobacter thermohydrosulfuricus; identical to SEQ ID NO: 11

SEQ ID NO: 16 - DNA, 30 bp, Artificial Sequence
  Description of Artificial Sequence Primer
ggcattggccacaccagccaccaccttctg

SEQ ID NO: 17 - DNA, 50 bp, Thermoanaerobacter thermohydrosulfuricus; identical to SEQ ID NO: 11

SEQ ID NO: 18 - DNA, 28 bp, Artificial Sequence
  Description of Artificial Sequence Primer
ggcattggccacaccagccaccaccttc

SEQ ID NO: 19 - DNA, 50 bp, Thermoanaerobacter thermohydrosulfuricus; identical to SEQ ID NO: 11

SEQ ID NO: 20 - DNA, 27 bp, Artificial Sequence
  Description of Artificial Sequence Primer
ggcattggccacaccagccaccacctt

SEQ ID NO: 21 - DNA, 50 bp, Thermoanaerobacter thermohydrosulfuricus; identical to SEQ ID NO: 11

SEQ ID NO: 22 - DNA, 27 bp, Artificial Sequence
  Description of Artificial Sequence Primer
ggcattggccacaccagccaccacctt

SEQ ID NO: 23 - protein, 40 aa, Thermotoga maritima
NVKPEEVTEEMRRAGKMVNFSIIYGVTPYGLSVRLGVPVK

SEQ ID NO: 24 - protein, 40 aa, Thermotoga neapolitana
NVKPEEVNEEMRRVGKMVNFSIIYGVTPYGLSVRLGIPVK

SEQ ID NO: 25 - protein, 40 aa, Thermotoga subterranea
GVSEMFVSEQMRRVGKMVNFAIIYGVSPYGLSKRIGLSVS

SEQ ID NO: 26 - protein, 40 aa, Escherichia coli
GLPLETVTSEQRRSAKAINFGLIYGMSAFGLARQLNIPRK

SEQ ID NO: 27 - protein, 40 aa, Thermus caldophilus
GVPPEAVDPLMRRAAKTVNFGVLYGMSAHRLSQELAIPYE

SEQ ID NO: 28 - protein, 40 aa, Thermus filiformis
GLDPALVDPKMRRAAKTVNFGVLYGMSAHRLSQELGIDYK

SEQ ID NO: 29 - protein, 40 aa, Thermus flavus
GVSPEGVDPLMRRAAKTINFGVLYGMSAHRLSGELSIPYE

SEQ ID NO: 30 - protein, 40 aa, Thermus thermophilus; identical to SEQ ID NO: 27

SEQ ID NO: 31 - protein, 40 aa, Thermus aquaticus
GVPREAVDPLMRRAAKTINFGVLYGMSAHRLSQELAIPYE

SEQ ID NO: 32 - protein, 40 aa, Thermoanaerobacter thermohydrosulfuricus
KVPMEKVTPEMRRAAKAVNFGIIYGISDYGLSRDLKISRK